(19) United States
(12) Patent Application Publication
(10) Pub. No.: US 2023/0346346 A1
(43) Pub. Date: Nov. 2, 2023

(54) APPARATUS, SYSTEM AND METHOD FOR FACILITATING ULTRASOUND MEDICAL IMAGING

(71) Applicants: MEDULLA PRO TECHNOLOGY PTE. LTD., Singapore (SG); TAN TOCK SENG HOSPITAL PTE LTD, Singapore (SG); ALEXANDRA HEALTH PTE. LTD., Singapore (SG)

(72) Inventors: Desmond Chuang Kiat SOH, Singapore (SG); Eric Chee Mun LOH, Singapore (SG); Wern Hsien BIN, Singapore (SG); Eugene Bingwen FAN, Singapore (SG)

(73) Assignees: MEDULLA PRO TECHNOLOGY PTE. LTD., Singapore (SG); TAN TOCK SENG HOSPITAL PTE LTD, Singapore (SG); ALEXANDRA HEALTH PTE. LTD., Singapore (SG)

(21) Appl. No.: 17/765,654

(22) PCT Filed: Aug. 21, 2020
(86) PCT No.: PCT/SG2020/050490

§ 371 Date: Mar. 31, 2022

(30) Foreign Application Priority Data
Aug. 23, 2019 (SG) 10201907817P

(51) Int. Cl. A61B 8/00

(52) U.S. Cl. CPC A61B 8/4209; A61B 8/4444; A61B 8/58

(57) ABSTRACT

The present disclosure relates to an apparatus (20) for facilitating medical imaging of a subject, comprising: a medical imaging device receiver configured to receive an ultrasound medical imaging device, a sound wave manipulation module comprising a soundwave deflection surface (113) arranged to direct transmission of sound waves between the ultrasound medical imaging device and the subject, and at least one auxiliary equipment receiver configured to receive an auxiliary equipment and to provide a pathway for insertion of the auxiliary equipment, wherein the pathway extends from the soundwave deflection surface to a base plane of the apparatus when rested on the subject, and an image of the subject is formed by the ultrasound medical imaging device for guiding insertion of the auxiliary equipment towards a target portion of the subject, and wherein the position of the pathway is calibrated for the ultrasonic medical imaging device being used (132). In particular, the auxiliary equipment is a needle, catheter or endoscope.

### FIELD

The present invention relates to an apparatus, system and method for ultrasound medical imaging.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Ultrasonography is a type of medical imaging technique which can be adopted in a variety of medical diagnosis and examination applications. Such diagnosis and examination applications include detection of tumors, providing images of fetuses for assessment of their development, and monitoring blood flow within various vital organs.

Ultrasonography has also been deployed to identify anatomy features of an individual such as a lumbar interspace of a vertebrate, such as, but not limited to, a human being. A known apparatus for use in ultrasonography is the wave guide, also known as an ultrasound probe. In the context of identifying a lumbar interspace, such waveguide apparatus typically operates on the principle of reflection of ultrasonic waves to identify the lumbar interspace before a mark is made on the outer skin of the person. A suitable equipment may then be inserted to the interspace using the mark for guidance. Such equipment may include, for example, a needle or catheter to administer local or general anesthetic.

In utilizing the waveguide and marker, it may be appreciated that both of the user's hands are utilized, i.e. one hand for holding and moving the wave guide to identify the lumbar interspace, the other hand for using a marker/identifier to mark the interspace area/spot on the skin. This may compromise the overall accuracy of the identification process as it depends on the user to ensure that the waveguide is not inadvertently shifted or moved when the user is marking the interspace area/spot on the skin.

An object of the invention is to ameliorate one or more of the above-mentioned difficulties.

### SUMMARY

According to one aspect of the disclosure, there is provided an apparatus for facilitating medical imaging of a subject comprising: a medical imaging device receiver configured to receive an ultrasound medical imaging device, a sound wave manipulation module comprising a soundwave deflection surface arranged to direct transmission of sound waves between the ultrasound medical imaging device and the subject, and at least one auxiliary equipment receiver configured to receive an auxiliary equipment and to provide a pathway for insertion of the auxiliary equipment, wherein the pathway extends from the soundwave deflection surface to a base plane of the apparatus when rested on the subject, and an image of the subject is formed by the ultrasound medical imaging device for guiding insertion of the auxiliary equipment towards a target portion of the subject, and wherein the position of the pathway is calibrated for the ultrasonic medical imaging device being used.

In some embodiments, a soundwave deflection surface is arranged to alter a direction of transmission of at least part of the sound waves.

In some embodiments, the soundwave deflection surface is configured to facilitate at least one of the following:—reflection, refraction, diffraction of sound waves.

In some embodiments, the soundwave deflecting surface is formed from a material having a sound transmission velocity at a ratio ranging from 1.0 to 11.0 relative to water.

In some embodiments, the soundwave manipulation module comprises a soundwave transmission portion arranged to facilitate transmission of the sound waves between at least the ultrasound medical imaging device and the soundwave deflection surface.

In some embodiments, the soundwave transmission portion is formed from a material having a sound transmission velocity at a ratio ranging from 0.8 to 5.0 relative to water.

In some embodiments, the soundwave transmission portion comprises a gel-based material.

In some embodiments, the gel-based material is disposed at a hollow portion of the sound wave manipulation module.

In some embodiments, the pathway of the auxiliary equipment receiver is provided by a tubular channel extending through the hollow section.

In some embodiments, the gel-based material is accommodated within a gel pad that protrudes through a calibration window provided at a base plane of the apparatus.

In some embodiments, the tubular channel extends to or through calibration window.

In some embodiments, the soundwave manipulation module is formed from a homogenous material having a sound transmission velocity at a ratio ranging from 1.0 to 3.0 relative to water.

In some embodiments, the sound wave manipulation module, the medical imaging device receiver and the auxiliary equipment receiver are integrally formed as a one-piece element.

In some embodiments, the at least one auxiliary equipment receiver is configured to facilitate placement of the auxiliary equipment at more than one desired positions with respect to a base plane of the apparatus.

In some embodiments, the ultrasound medical imaging device is mounted at a first angle between 0 to 90 degrees with respect to a base plane of the apparatus, and the sound wave deflection surface may be mounted at a second angle between 0 to 90 degrees with respect to a base plane of the apparatus.

In some embodiments, the apparatus comprises a handle for controlling movement of the apparatus on a body surface of the subject.

In some embodiments, the apparatus comprises a locking mechanism for maintaining the medical device at a desired position relative to a part of the apparatus.

In accordance to another aspect of the disclosure, there is provided a method for deploying an apparatus for facilitating medical imaging. The method comprises the steps of:—attaching an ultrasound medical imaging device to the medical imaging device receiver of the apparatus; placing the apparatus on a body surface of a subject; moving the apparatus on the body surface for obtaining an image of a target portion of the subject; attaching an auxiliary equipment to the at least one auxiliary equipment receiver of the apparatus; adjusting position of the auxiliary equipment along the pathway provided by the at least one auxiliary equipment receiver based on the image of the target portion, and inserting the auxiliary equipment towards the target portion.

According to a further aspect of the present invention, there is provided a process of manufacturing an apparatus as described above, comprising inserting the gel pad into the hollow section of the sound transmission module such that a portion of the gel pad protrudes through the calibration window.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention, wherein

FIG. 1 show an apparatus for facilitating medical imaging in use with a medical imaging device and an auxiliary equipment mounted thereon in accordance with one embodiment;

FIGS. 2 and 3 show an apparatus for facilitating medical imaging in accordance to another embodiment;

FIGS. 4A and 4B show an apparatus for facilitating medical imaging in accordance with another embodiment;

FIGS. 5A to 5D respectively show a perspective, bottom and two rear views of an apparatus for facilitating medical imaging in accordance with a further embodiment;

FIGS. 6A to 6C respectively show perspective, bottom and rear views of an apparatus for facilitating medical imaging in accordance with another embodiment;

FIGS. 7A to 7C respectively show perspective, bottom and rear views of an apparatus for facilitating medical imaging in accordance with yet another embodiment;

FIG. 8 is a schematic view showing the calibration of the pathway of the auxiliary equipment receiver; and

FIG. 9 depicts a method of using the apparatus according to some embodiments.

### DETAILED DESCRIPTION

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the specification, unless the context requires otherwise, the word “include” or variations such as “includes” or “including” will be understood to imply the inclusion of a stated integer or group of integers but not the exclusion of any other integer or group of integers.

Throughout the specification, the term ‘medical image’ or ‘medical imaging’ may include images or imaging methods based on a variety of techniques and include the process of creating visual representations of an interior of a body for clinical analysis and medical intervention, as well as visual representation of the function of some organs or tissues.

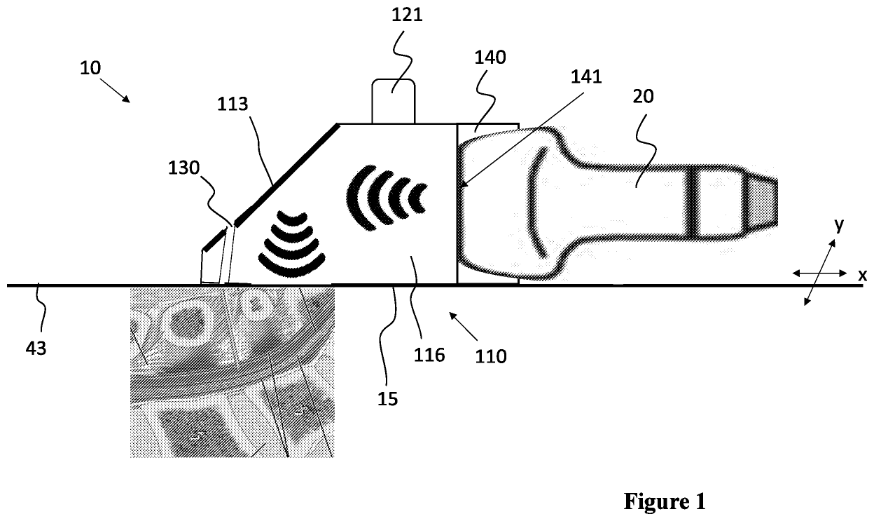

According to one aspect of the invention and with reference to FIGS. 1 to 5B there is an apparatus 10 for facilitating medical imaging of a subject, for example a body portion of a patient. The apparatus 10 comprises a medical imaging device receiver 140 configured to receive a medical imaging device 20, a sound wave manipulation module 110 arranged to direct transmission of sound waves 115 between the medical imaging device 20 and the subject along a designated path, and at least one auxiliary equipment receiver 130 configured to receive an auxiliary equipment (not shown) for placement at a target portion of the subject, wherein an image of the target portion is formed by the medical imaging device 20 for guiding placement of the auxiliary equipment.

In various embodiments, the medical imaging device 20 may be in the form of an ultrasound probe 20. An exemplary ultrasound probe may include a transducer for producing sound waves of a specific frequency range, which are focused either by the shape of the transducer, a lens in front of the transducer, or a complex set of control pulses from a transmit beam-former coupled to the transducer. An arc-shaped sound wave is transmitted from the face of the ultrasound probe 20 into a target subject (for example, a patient). The waveform and frequency of the sound waves may be adapted such that the sound waves may travel along one or more ultrasound scan lines and may travel into the target subject at a desired depth. The transducer of the ultrasound probe is operable to receive echoes of the sound waves from the target subject. The ultrasound probe may further comprise or may be connected to image processing modules/circuits which are operable to interpret the received echoes data to generate an image of the target subject.

It is to be appreciated that that alternative techniques of generating and controlling ultrasound waves as well as receiving and interpreting echoes received therefrom for the purpose of diagnostic medical imaging may also be used with the various embodiments of the present disclosure. For example, other types of transmitters and/or receivers may be used in addition to or in substitution of the transducer, which may eliminate the need for a transmit beamformer, and may permit beam forming to be performed by post processing the received echoes. It is also appreciated that various signal processing techniques may be performed on the received echoes. For example, a receive beamformer and/or various digital/analog signal processing techniques may be used to acquire image information from the received sound wave echoes and to perform three-dimensional image reconstruction from a plurality of two-dimensional image planes of the target subject.

In various embodiments, the auxiliary equipment 22 may be a tool for assisting a medical diagnostic procedure. In some embodiments, the auxiliary equipment may be an invasive medical device including, but not limited to, an aspiration or biopsy needle, a catheter, and an endoscope. In use, a clinical practitioner needs to place or insert the auxiliary equipment 22 towards specific targets at/inside a body portion of the subject. It is appreciated that accuracy and speed of placement/insertion of the auxiliary equipment can be critical in such procedures.

In various embodiments, the apparatus 10 comprises a medical imaging device receiver 140 for receiving and/or holding the medical imaging device 20.

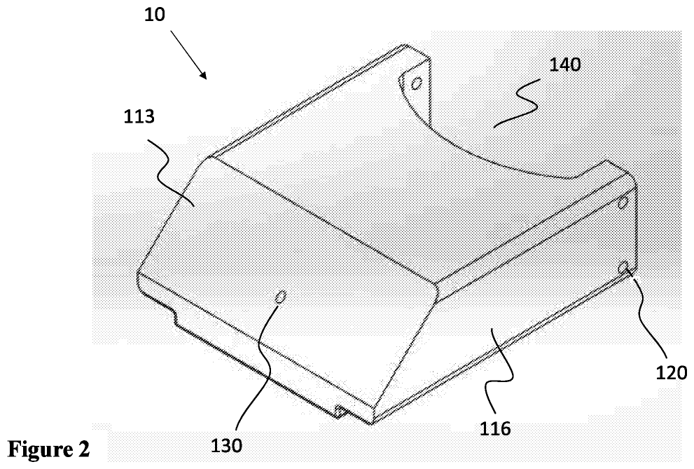

In some embodiments as shown in FIG. 1, FIG. 2 and FIGS. 4 to 5B, the apparatus 10 may comprise a casing for containing components of the apparatus including the sound wave manipulation module 110. The casing of the apparatus may be provided with a structure (e.g. a moulded plastic part) shaped and dimensioned as a medical imaging device receiver 140 for receiving the medical imaging device 20. In use, the medical imaging device 20 (e.g. an ultrasound probe) may be plugged or fitted into the medical imaging device receiver 140. The medical imaging device receiver 140 may be adaptable or comprise adaptable structures (e.g. adjustable sides, slidable portions) to receive most, if not all commercially available ultrasound probes.

In some embodiments, the medical device receiver 140 may comprise a disposable interface (not shown). The disposable interface can include an interface attachment and may be an area/feature where sterility is to be maintained.

In some embodiments, the disposable interface contains or comprises of various materials such as polymers. Such materials are single-use and disposable because of the nature of the material, and its limited shelf life. In addition, when used with any ultrasound gel and the ultrasound probe, some wear and tear would render this part unusable or not economical or not easy to clean, prepare and re-use.

In some embodiments, the medical device receiver 140 may be further provided with a locking mechanism 120 for securing/maintaining the medical imaging device 20 at a desired position with respect to the apparatus 10. One non-limiting example of the locking mechanism 120 may be a snap fastener for locking a corresponding protruding edge on the medical imaging device 20. It is to be appreciated that various types of snap fastener including annular, torsional, cantilever snap fit designs may be implemented as the locking mechanism 120 for securing the medical imaging device 20. Loose movement of the medical imaging device 20 with respect to the apparatus 10, which may introduce noises/interferences to the imaging system, may be reduced by using the locking mechanism 120.

In various embodiments, the apparatus 10 comprises a soundwave manipulation module 110 arranged to direct transmission of sound waves between the medical imaging device 20 and the subject (more specifically, a body portion of the subject where the apparatus 10 is placed on). In particular, the soundwave manipulation module 110 includes one or more acoustic components that are capable of changing at least the transmission direction of the sound waves.

In various embodiments, the soundwave manipulation module 110 may include a soundwave deflecting surface 113 and a soundwave transmission portion 116. A deflector material may be provided to achieve function of deflecting or re-directing the soundwaves at the soundwave deflecting interface 113. A sound transmission material may be used for forming the soundwave transmission portion 116 so as to facilitate transmission of the sound waves therein.

In various embodiments, the soundwave deflection surface 113 may be arranged to alter a direction of transmission of at least part of the sound waves. In use, the medical imaging device 20 (e.g. the ultrasound probe) is placed next to or immediately adjacent the soundwave manipulation module 110 so that ultrasound waves produced from the medical imaging device 20 are directed to travel towards the soundwave deflection surface 113.

In various embodiments, the medical imaging device may be mounted at a first angle between 0 to 90 degrees with respect to the base plane 15 of the apparatus 10, and the soundwave deflection surface 113 may be arranged at a second angle between 0 to 90 degrees with respect to the base plane 15 of the apparatus 10. In use, the base plane 15 of the apparatus may be rested on the surface 43 of the body portion of the subject (i.e. the body surface 43), or may be substantially parallel with and close to the body surface 43. The medical imaging device 20 is not firing acoustic energy or transmitting the sound waves directly towards the body surface 43, when the apparatus 10 (with the medical imaging device 20 attached thereon) are placed on the body surface 43.

The soundwave deflection surface 113 functions as a soundwave re-director that allows the sound waves to travel towards the body portion of the subject, and allows the echoes from the body portion to travel back to the ultrasound probe for image construction. The transmission of the sound waves follows a designated path or a designated propagation trajectory. More specifically, as can be seen in FIG. 1, the sound waves propagate from the ultrasound probe 20 to the soundwave deflection surface 113 in a first direction substantially along a base plane 15 of the apparatus 10. The soundwave deflection surface 113 may be positioned at an angle with respect to the base plane 15. As the sound waves (e.g. in the form of longitudinal acoustic arrays) strikes the soundwave deflection surface, at least part of the sound waves are deflected to propagate along a second direction and towards a body portion of the subject where the base plane 15 of the apparatus 10 is rested on. Soundwave echoes from the body portion of the subject are transmitted towards the soundwave deflection surface 113 and are redirected to propagate to towards the ultrasound probe receptor along a similar or the same propagation trajectory.

In various embodiments, the soundwave deflection surface 113 may be configured to work based on principles of wave reflection, wave refraction, or wave diffraction. In particular, there are several ways to redirect the ultrasound waves. These include:—a. diffraction as the ultrasound waves pass through one or more openings (grating) if present or around a barrier; b. refraction as the ultrasound waves pass through material of different properties, i.e. through layers of dissimilar or inhomogeneous media. Examples of such properties may be density of the material, a sound wave impedance or a sound wave transmission velocity of the material.

In some embodiments, the soundwave deflection surface 113 is formed from a deflector material having a sound transmission velocity substantially different from the sound transmission material of the soundwave transmission portion 116. Alternatively, the sound transmission velocity of the deflector material may be in a substantially different range as compared to that of the soundwave transmission material. In some embodiments, suitable deflector material may have a sound transmission velocity at a ratio ranging from 1.0 to 11.0 relative to water.

Deflection of the sound waves occurs at an interface of two dissimilar medium, i.e. the deflector material and the sound transmission material. Due to the differences in the sound wave transmission velocity of the deflector material and of the sound transmission material, the sound waves are deflected at the soundwave deflection surface. In this manner, the propagation path/trajectory of the sound waves may be altered. It is to be appreciated that a desired degree of change in the propagation direction (i.e. the degree of deflection of the sound waves) may be achieved by selecting a suitable deflector material, and/or by positioning and shaping the soundwave deflection surface 113 in a suitable manner.

In some embodiments, for the purpose of effective soundwave deflection, one or more reflective surface(s) may be provided to substitute or supplement the deflector material. The reflective surface(s) can be fabricated out of suitable materials for reflecting ultrasound waves coherently and efficiently. The shape or texture of the reflective surface(s) may also be designed to reshape or focus the wave pattern so as to improve clarity or efficiency of wave reception through soundwave manipulation.

In some embodiments, the reflective surface(s) may comprise one or more rigid materials such as polypropylene (PP), polycarbonate (PC), glass, metal or suitable polymers. The following modifications may be made to one or more of the aforementioned material(s):—Suitable coating materials may be chemically deposited or electroplated with various metals such as gold, nickel, copper, chrome, etc. The reflective surface may comprise or predominantly consist of microstructures or patterned textures to manipulate the ultrasound waves so as to achieve proper focusing or beam forming. The reflective surface may be constructed of porous or non-porous internal structure of the materials mentioned.

In some embodiments, the reflective surface(s) may be supplemented by one or more diffraction mechanisms such as ultrasonic acoustic grating. In some embodiments, multiple reflective surfaces may be arranged at various angles with respect to each other for the optimal reflection.

In some embodiments, the soundwave deflection surface 113 may be a replaceable part. Different configuration or different wave forming feature may be mounted onto the apparatus 10 and be deployed as the soundwave re-director. For example, a replaceable part, which will influence the pattern of the sound wave differently, may be used to generate the image in a more efficient manner for different thickness of the skin or body structure.

In various embodiments, the soundwave manipulation module 110 may further comprise a soundwave transmission portion 116 arranged to facilitate efficient transmission of the sound waves within the soundwave manipulation module 110. More specifically, the soundwave transmission portion 116 is arranged as a medium for the soundwaves to travel between the medical imaging device 20 (e.g. the ultrasound probe transmitter/receptor end) and the soundwave deflection surface 113, as well as between the soundwave deflection surface 113 and the target portion of the subject, in accordance to the designated transmission or propagation trajectory controlled by the soundwave deflection surface 113.

Suitable sound transmission material(s) having a desired acoustic characteristic (e.g. a desired sound transmission velocity) may be used for forming the soundwave transmission portion 116. In some embodiments, the sound transmission material(s) may have a sound transmission velocity at a ratio ranging from 0.8 to 5.0 relative to water. In some embodiments, the sound transmission materials may have a sound transmission velocity at a ratio ranging from 1.0 to 3.0 relative to water.

In various embodiments, the soundwave transmission module may be formed with a clear and homogeneous structure. The structure may be transparent or translucent. Artifacts within the soundwave transmission portion 116 are minimized or eliminated so as to facilitate efficient transmission of the sound waves therein.

In various embodiments, suitable sound transmission materials for forming the soundwave transmission portion 116 includes, but are not limited to Poly(methyl methacrylate) or PMMA, Polycarbonate or PC, Polyamide (e.g. Nylon), Polyvinyl chloride or PVC, Polystyrene or PS, Polypropylene or PP, silicone or polysiloxanes, natural or synthetic rubber.

In some embodiments, the soundwave transmission portion 116 may be formed from a water-based material or a gel-based sound transmission material which results in the travel speed of ultrasound resembling that of water or a soft tissue. Such water-based or gel-based sound transmission materials may include, but are not limited to water, gelatine, polyvinyl alcohol, agarose, and polyacrylamide. The water-based or gel-based soundwave transmission material may be provided in the form of a gel pad. The gel pad may be disposed at a hollow portion or a cavity of the soundwave manipulation module 110. The form and shape of the gel pad soundwave transmission material are adaptable according to the inner profile of the hollow portion/the cavity of the soundwave manipulation module 110. Advantageously, any gaps, air pockets, or other irregularities which may interfere the sound wave propagation within the soundwave transmission portion 116 (in the form of a gel pad disposed in the hollow portion) can be minimized.

In various embodiments, the soundwave manipulation module 110 may be supplemented by one or more interface materials. In use, the one or more interface materials may be disposed at an interface between the medical imaging device 20 and the sound wave transmission portion 116 (i.e. the probe-apparatus interface 141) and/or at an interface between the sound wave transmission portion 116 and the body portion of the subject.

Similar to the sound transmission material used for forming the gel-pad form soundwave transmission portion 116, the primary (core) component of the interface material(s) may be water-based (e.g., gelatine, polyvinyl alcohol, agarose, polyacrylamide) which results in the travel speed of ultrasound resembling that of water or soft tissue. In addition, a scattering agent may be suspended in the buffer/gel medium to produce the backscatter that enhances ultrasound imaging. Scattering agents generally comprise particulate matter and may include graphite particles, silica particles, and polystyrene spheres.

The interface material(s) may be provided to improve efficiency and compatibility with existing ultrasound probe (i.e. for ultrasound buffer). Such interface material(s) can include acoustic materials including gelatine-based material (i.e. gel) with various additives to provide realistic acoustic properties to enhance or control ultrasonic (US) waves. The additives may be micron-sized silica particles or similar to induce acoustic scattering and a percentage (range) of fat emulsion to change ultrasonic attenuation. It is to be appreciated that in general the interface material can be modified to achieve an optimum or optimum range of speed of ultrasound travelling through a medium, acoustic attenuation and acoustic backscatter.

In some embodiments, the soundwave transmission portion 116 may further comprise a buffer material that enhances the soundwave transmission efficiency through the various mediums to the ultrasound probe receptor by providing an interface with the feature. This buffer material may be a disposable part which is connectable to and compatible with various probe shapes.

In various embodiments, the apparatus 10 comprises at least one auxiliary equipment receiver 130 for receiving the at least one auxiliary equipment. The auxiliary equipment may be disposed on or otherwise connected to the soundwave deflection surface 113 of the soundwave manipulation module 110.

In various embodiments, the auxiliary equipment receiver 130 is shaped and dimensioned to receive an auxiliary equipment for insertion towards and/or into the body portion of the subject. The auxiliary equipment receiver 130 provides a means for holding and/or guiding the auxiliary equipment. For example, as shown in FIGS. 1 to 4, the auxiliary equipment receiver 130 may be in the form of an aperture/channel shaped and dimensioned for the auxiliary equipment (such as a needle or a catheter) to be inserted. The auxiliary equipment receiver 130 may be configured to receive most, if not all commercially available aspiration/biopsy needles and catheters.

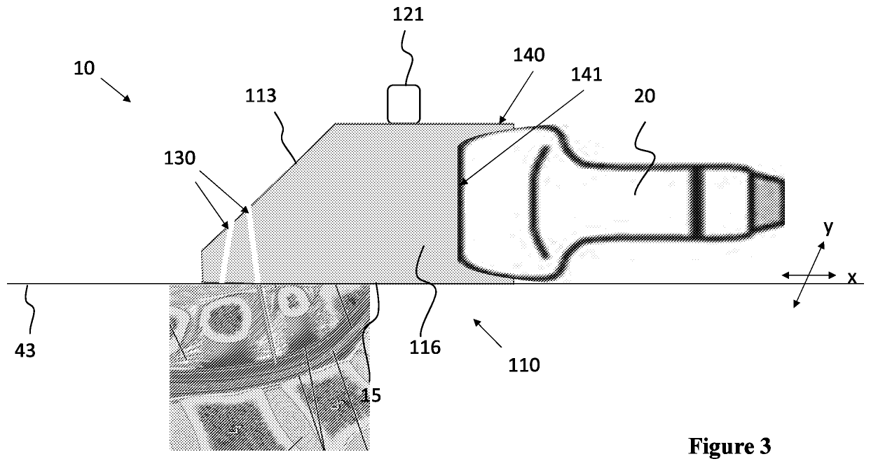

In some embodiments, the at least one auxiliary equipment receiver 130 may be configured to facilitate placement of the auxiliary equipment at more than one positions with respect to the base plane 15 of the apparatus 10. For example, the apparatus 10 may be provided with more than one aperture/channel of different shapes and dimensions for receiving auxiliary equipment of different types. Further, as illustrated in FIG. 3, the apertures/channels for receiving the auxiliary equipment may be arranged at different angles with respect to the base plane 15 of the apparatus 10. This allows placement/insertion of the auxiliary equipment from different directions, with reference to the base plane of the apparatus 15 or the body surface 43.

The shape and dimension of the aperture/channel may correspond substantially to the shape and dimension of the needle or the catheter, which allows insertion of the needle or the catheter along a longitudinal axis of the aperture/channel, and at the same time may allow lateral or rotational movement of the needle/catheter within the aperture/channel to a certain degree. In other words, the aperture/channel and the auxiliary equipment are not in a tight-fit arrangement. When the needle/catheter is inserted into the aperture/channel, a gap is left between the needle/catheter and the inner wall of the aperture/channel so that is the needle/catheter is not completely confined by the aperture/channel and the position of the needle/catheter is adjustable to a certain degree. In some embodiments, the inserted needle or catheter is capable of an angular movement and/or a lateral movement within the aperture/channel. For example, the needle/catheter held by the aperture/channel may be capable of an angular movement of −20° to 20° about the central axis of the aperture or about the longitudinal axis of the channel.

In some embodiments, the apparatus 10 further comprises a handle for controlling movement of the apparatus on the body surface 43 of the subject. For example, the handle 121 may be a protruding part provided on an upper surface of the apparatus 10, as shown in FIGS. 1 and 3. Particularly, the handle 121 provides convenience to the user (e.g. a medical practitioner) to move the apparatus 10 across the body surface 43 to identify the target portion.

In some embodiments, the medical imaging device receiver 140 and the at least one auxiliary equipment receiver 130 are rotatable with respect to each other to achieve an optimal view of an auxiliary equipment (when present) and a clear pathway to the target subject. More specifically, the ultrasound probe 20 and/or the auxiliary equipment receiver 140 may be rotatable about a portion (point) on the base plane 15 of the apparatus 10 to provide for angular adjustment. A practitioner may adjust the position of the ultrasound probe 20 and the auxiliary equipment receiver 40 to obtain an optimal ultrasound image.

Advantageously, the afore-described apparatus 10 provides for an arrangement to redirect ultrasound waves effectively by reflection or diffraction techniques to provide a clear view of the auxiliary equipment such as needle (when present) for insertion into an identified location on a subject. The arrangement also provides for a clear pathway and an optimal view of the auxiliary equipment to the identified location of the subject.

Further, by referring to a real-time image of the target portion formed by the medical image device, a practitioner may accurately place the auxiliary equipment at or insert the auxiliary equipment into the target portion of the subject. The step of marking a location for placement/insertion of the auxiliary equipment, and removing the ultrasound probe to prepare for placing/inserting the auxiliary equipment into the target portion may be eliminated.

It is to be appreciated that the arrangement of the various components of the apparatus 10 as described may implemented in various suitable manners.

In some embodiments as illustrated in FIG. 1 and FIG. 2, one both of the auxiliary equipment receiver 130 and the medical imaging device receiver 140 may be formed as part of the casing of the apparatus 10. For example, these features may be integrally formed with the casing of the apparatus using a plastic molding technique. The casing may comprise adaptable structures for placement and/or attachment of other components of the apparatus 10 including the soundwave deflection surface 113 and the soundwave transmission portion 116.

In some other embodiments, as illustrated in FIG. 3, the apparatus 10, in particular, the sound wave manipulation module 110, the medical device receiver 140 and the auxiliary equipment receiver 130 may be integrally formed as a one-piece element, for example, by using a plastic moulding process. Where necessary, one or more other shaping processes may be used to form structures for mounting/receiving the medical imaging device 20 and the auxiliary equipment. Such an apparatus 10 may be referred to as a “mono-block” design or a “mono-block” apparatus 10.

In various embodiments, mono-block apparatus 10 is shaped and dimensioned such that the soundwaves propagating within the mono-block apparatus along a designated path. In particular, the soundwave deflecting surface 113 may be disposed at a suitable angle for re-directing the soundwaves towards the target body portion or re-directing the soundwave echoes towards the medical imaging device 20 (or ultrasound probe). Further, the mono-block apparatus 10 may be formed to extend along a suitable length from the probe-apparatus interface 141 to the soundwave deflecting surface 113, for example, in a range of 5 mm to 60 mm.

As can be seen from FIG. 1 and FIG. 2, in the mono-block design, an interface between the mono-block material and the air forms and functions as the soundwave deflection surface 113.

Materials with a suitable sound transmission velocity, which allows the trajectory of sound waves to be deflected at a desired angle at the sound deflection surface 113, may be used for forming the mono-block apparatus 10. Such materials may include the afore-described sound transmission materials, such as Poly(methyl methacrylate) or PMMA, Polycarbonate or PC, Polyamide (e.g. Nylon), Polyvinyl chloride or PVC, Polystyrene or PS, Polypropylene or PP, silicone or polysiloxanes, natural or synthetic rubber.

In various embodiments, the materials forming the mono-block apparatus 10 may have a desired flexibility. When placed on the body surface 43, the apparatus 10 (in particular, the base plane 15 of the apparatus 10) may then better conform to the body contour. Air pockets or any gaps between the apparatus 10 and the body surface 43 are reduced or minimized. Image quality is further improved. The step of applying interface materials (such as ultrasound gel) on the body surface for sealing the air pockets/gaps may be eliminated. Such ultrasound gel may cause inconvenience to the patients and may raise sterility issues, which are undesirable.

In some embodiments, the material used for forming the mono-block apparatus 10 may have a sound transmission velocity at a ratio ranging from 1.0 to 3.0 relative to water. Accordingly, sound waves that are transmitted from the medical imaging device 20 and echoes that come from the body surface may be deflected towards the mono-block material at a desired angle when striking on the soundwave deflection surface 113.

In various embodiments, the mono-block apparatus 10 may be formed with a clear and homogeneous structure, where artifacts (such as air pockets, impurities) are minimized or eliminated with the apparatus so as to minimize interference to the sound waves travelling therein. The soundwave deflecting surface 113 and the probe-apparatus interface 141 are smooth and homogenous surfaces formed with minimal surface roughness/irregularities. Advantageously, efficient soundwave transmission within the mono-block apparatus 10, and efficient soundwave diffraction at the soundwave deflecting interface 113 may be achieved.

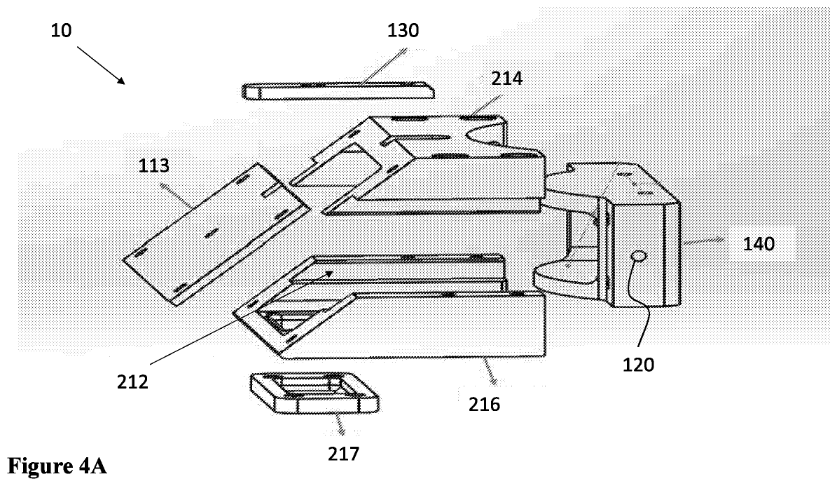

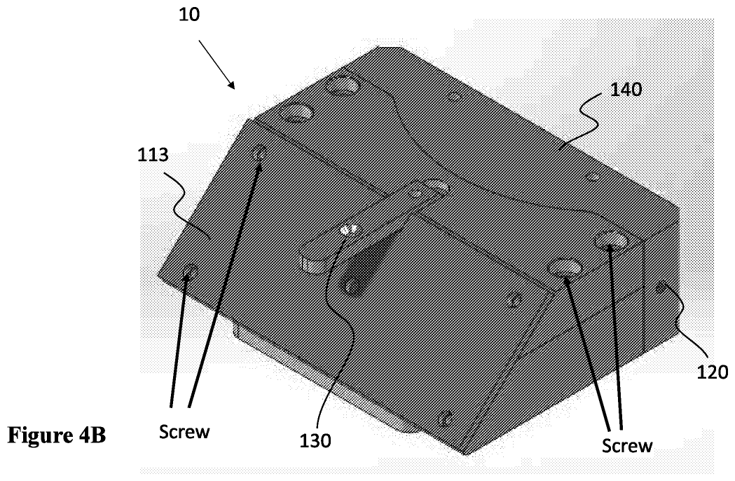

In some embodiments as illustrated in FIGS. 4A and 4B, the auxiliary equipment receiver 130, the casing, and other components of the apparatus including the soundwave deflection surface 113, the medical imaging device receiver 140, and the soundwave transmission module 116, may be formed as separate parts which are assembled/connected together. As can be seen, a hollow portion 212 may be formed by a top block 214, a bottom block 216 and a catch 217 when assembled together. The hollow portion 212 may be used for containing a soundwave transmission material, e.g. a gel pad containing a gel-based material. Multiple attachment means (e.g. by using screws, rivets, adhesive material(s), mechanical interlocking structures) may be used for connecting/assembling the aforementioned parts to form the apparatus 10.

The gel-based material is a flexible and soft material that conforms to the body contour to reduce/eliminates air pockets between the apparatus-body interface and thus provide for better imaging quality. Also, as the gel-based material is an excellent sound transmission material, the step of applying interface materials (such as ultrasound gel) on the body surface for sealing the air pockets/gaps may be eliminated. Inconvenience for the patients may be reduced and sterility issues caused by using such interface materials may be avoided.

In this configuration, the soundwave deflection surface 113 and other components of the apparatus may be replaceable. A suitable soundwave deflection surface 113 may be selected based on the specific application of the apparatus, for example, based on the required image resolution and focus. Also, broken or worn parts may be replaced.

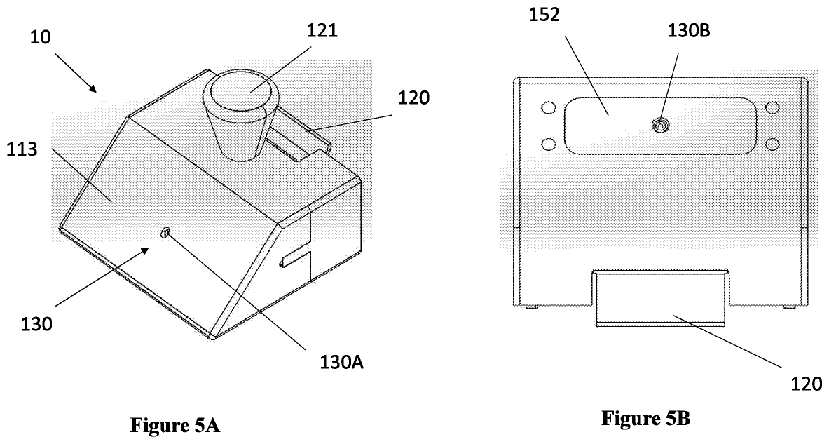

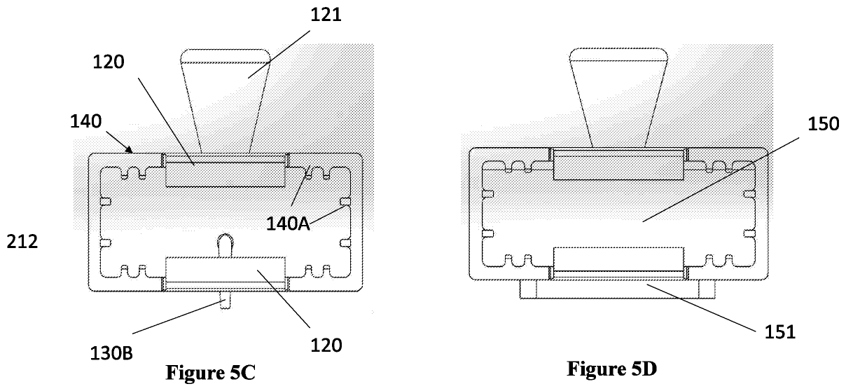

FIGS. 5A to 5D show another embodiment of an apparatus 10 according to the present disclosure. This apparatus 10 is similar to the embodiment shown in FIGS. 4A and 4B in having a hollow portion 212 within which can be accommodated therein a gel pad 150. The apparatus 10 further has a soundwave deflection surface 113, a handle 121 for facilitating movement of the apparatus 10 over the body surface 43, and a locking mechanism 120 in the form of a snap-fit locking mechanism for holding in position the medical imaging device 20 when secured to the apparatus 10. Two opposing locking mechanisms 120 may be provided about the periphery of the receiver 140 at the top and bottom portions thereof. The medical imaging device receiver 140 further includes a series of receiver grip portions 140A for minimizing the movement of the medical imaging device 20 when held by the medical device receiver 140. The medical imaging device 20 can then be held against the gel pad 150 allowing ultrasound wave from the device 20 to be transmitted into the gel within the gel pad 150.

The auxiliary equipment receiver 130 is supported by the soundwave deflection surface 113. A funneled opening 130A of the auxiliary equipment receiver 130 provides an entrance through the soundwave deflection surface 113 to a tubular channel 130B extending from the soundwave deflecting surface 113. The tubular channel 130B passes through the hollow portion 212 to a calibrated window 152 provided on the base plane 15 of the apparatus 10. A protrusion 151 of the gel pad 150 accommodated within the hollow portion 212 extends through the calibrated window 152. This gel pad protrusion 151 will help to ensure that the gel pad 150 is in contact with the body surface 43 of the patient. Furthermore, this eliminates the need for additional ultrasound gel to be spread on the body surface 43, so that it is not necessary to wipe excess gel from the body surface 43 after use of the apparatus 10. Furthermore, the potential of ultrasound gel entering the patient body through the puncture made by the auxiliary equipment is avoided. The funneled opening 130A helps to facilitate the insertion of auxiliary equipment such as a needle or catheter into the tubular channel 130B of the auxiliary equipment receiver 130. The tubular channel 130B then helps to guide the auxiliary equipment to the body surface 43 minimizing any misalignment of the auxiliary equipment.

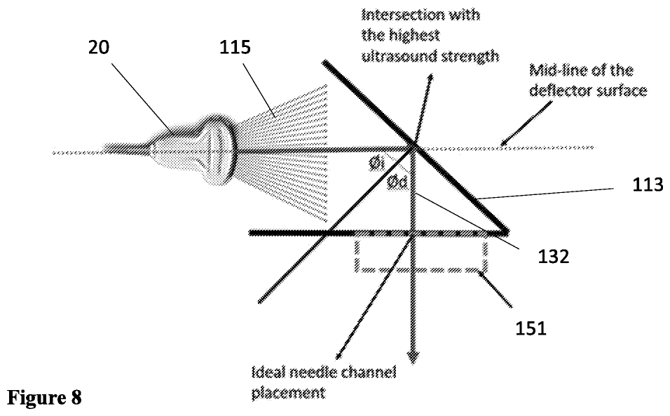

The position of the tubular channel 130B and calibration window 152 is calibrated for the ultrasonic medical imaging device 20 being used by the general direction of propagation of ultrasound waves from the ultrasonic medical imaging device 20 as shown in FIG. 8. The incident angle (Øi) of the propagated ultrasound waves relative to the soundwave deflection surface 113, and the deflection angle (Ød) of the ultrasound waves 115 reflected from the soundwave deflection surface 113 may be determined, where Øi=Ød is preferably obtained. This information can then allow for calibration of the position of the pathway 132 of the auxiliary equipment receiver 130 and the calibration window 152. This helps to ensure that the insertion point of the auxiliary equipment corresponds to ultrasound image that has been captured by the medical imaging device 20.

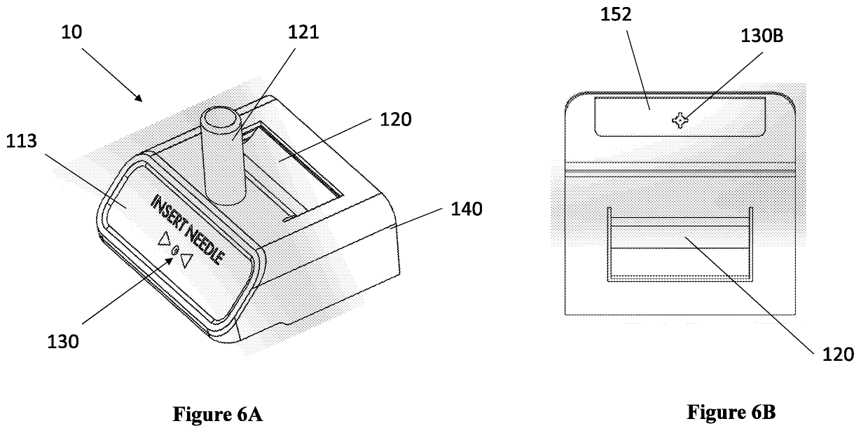

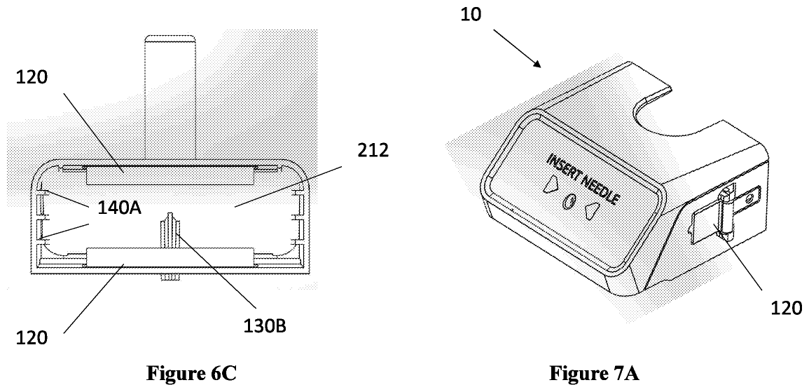

The apparatus 10 shown in FIG. 6A to 6C is similar to the embodiment shown in FIGS. 5A to 5D in having a soundwave reflection surface 113 from which is supported an auxiliary equipment receiver 130. A tubular channel 130B of the auxiliary equipment receiver 130 similarly extends through the hollow portion 212 to the calibrated window 152. A handle 121 is also provided extending from the upper wall of the apparatus 10. This embodiment however differs in the apparatus 10 having a deeper hollow section 212 that allows the medical imaging device 20 to be more deeply inserted into the hollow section 212. Opposing locking mechanisms 120 in the form of a snap-fit locking mechanism are provided within the upper and lower walls of the apparatus 10. These can engage the medical imaging device 20 when inserted through the receiver 140 into the apparatus 10. Receiver grip portions 140A are also provide around the entrance of the receiver 140 to minimize movement of the medical imaging device 20 when accommodated therein.

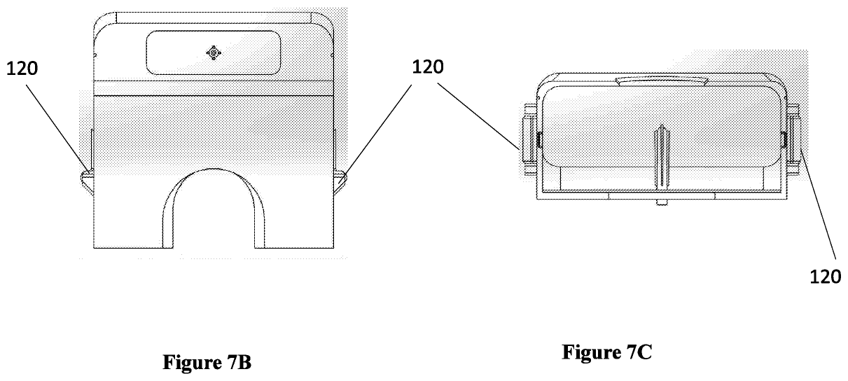

The embodiment of the apparatus 10 shown in FIGS. 7A to 7C is similar to the embodiment shown in FIGS. 6A to 6C, the primary difference being that the locking mechanism 120 is in the form of opposing clamping locking mechanisms provided on the opposing side walls of the apparatus 10. Furthermore, no handle is provided on this embodiment of the apparatus 10.

There is also provided a process of manufacturing an apparatus 10 according to the present disclosure which comprises a sound wave transmission portion 116 having a hollow portion 212 therein as is the case with the embodiments of the apparatus 10 shown in FIGS. 5A to 7C. The sound wave transmission portion 116 can be held in a jig while a movable fixture may insert the gel-pad 150 though the opening provided by the medical imaging device receiver 140. The gel-pad 150 can be inserted with a portion of the gel-pad 150 protruding through the calibration window 152 to form the gel-pad protrusion 151 as shown in FIG. 5D.

It is to be appreciated that the afore-described arrangements of the various components of the apparatus 10 are non-limiting examples, and other suitable arrangements may be contemplated to achieve the same effect of re-directing the sound waves using a sound manipulation module, allowing a real-time image of the subject to be formed for guiding the insertion/placement of an auxiliary equipment.

The apparatus 10 is described in the context of a method for facilitating medical imaging. The method may suitably be deployed to identify a lumbar interspace of an individual but it is to be appreciated that the method may be deployed for other types of medical imaging as known to a skilled person.

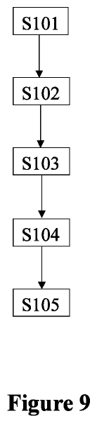

In accordance to one embodiment as shown in FIG. 9, the method for deploying the apparatus 10 may comprise the following steps:—

The medical imaging device 20 is first attached to the apparatus 10 via a medical imaging device receiver 140 (step s101). The sound wave manipulation module 110 is then placed on a body portion of a subject, e.g. on a back of the individual, preferably by a qualified medical practitioner (step s102). The base plane 15 of the apparatus 10 may be rested on the body surface 43.

Position of the apparatus 10 can be adjusted for obtaining an image of the target subject (step s104) by the medical imaging device 20 (step s103). The medical imaging device 20, i.e. the ultrasound probe is switched on and the apparatus 10 may be moved on and across the body surface 43, whereby a target portion may be identified for further procedures to be performed by the auxiliary equipment. For example, a user may move the apparatus 10 and the ultrasound probe 20 to locate the lumbar interspace of the subject, which can be, but not limited to, an L2-L3 interspace, L3-L4 interspace, L4-L5 interspace.

An auxiliary equipment (e.g. a needle, a catheter or an endoscope) can be attached to the apparatus 10 via the at least one auxiliary equipment receiver 130 (step s104). The apparatus 10 may comprise more than one auxiliary equipment receiver 130, and a suitable auxiliary equipment receiver 130 may be selected primarily based on the size and shape of the auxiliary equipment.

Once the target portion is identified, the auxiliary equipment may be inserted towards the target portion. The real-time image formed by the medical imaging device 20 may be used to guide the placement/insertion of the auxiliary equipment (step s105). In this process, the position of the auxiliary equipment may be adjusted based on the rea-time image of the target portion. In this manner, accurate and fast placement of the auxiliary equipment is achieved.

An apparatus for use with a medical imaging device, such as an ultrasound waveguide device has been contemplated as described in the present disclosure. The medical imaging device and one or more auxiliary equipment holder are arranged at various positions with respect to one another to achieve an optimal view of an auxiliary equipment (when present) and pathway to a target portion of a subject (e.g. a patient).

It should be appreciated by the person skilled in the art that the above invention is not limited to the embodiments described. In particular, modifications and improvements may be made without departing from the scope of the present invention.

It should be further appreciated by the person skilled in the art that one or more of the above modifications or improvements, not being mutually exclusive, may be further combined to form yet further embodiments of the present invention.

### REFERENCE

10 apparatus
15 base plane
20 medical imaging device
40 target subject
43 body surface
110 sound wave manipulation module
113 sound wave deflection surface
116 sound wave transmission portion
120 locking mechanism
121 handle
130 auxiliary equipment receiver
130A funnelled opening
130B tubular channel
132 auxiliary equipment receiver pathway
134 needle holder
140 medical imaging device receiver
140A receiver grip portion
141 probe-apparatus interface
150 gel pad
151 gel pad protrusion
152 calibration window
212 hollow portion
214 top block
215 bottom block
216 sound wave transmission portion
217 catch

1. An apparatus for facilitating medical imaging of a subject comprising:
a medical imaging device receiver configured to receive an ultrasound medical imaging device adapted to transmit sound waves in a first direction of transmission,
a sound wave manipulation module comprising a soundwave deflection surface arranged to alter the transmission of at least part of the sound waves from the ultrasound medical imaging device to the subject from the first direction of transmission to a second direction of transmission, and
at least one auxiliary equipment receiver configured to receive an auxiliary equipment and to provide a channel for insertion of the auxiliary equipment, wherein the channel extends through the sound wave manipulation module from the soundwave deflection surface to a base plane of the apparatus when rested on the subject, and an image of the subject is formed by the ultrasound medical imaging device for guiding insertion of the auxiliary equipment towards an insertion point in a target portion of the subject, and wherein the position of the channel is calibrated by being aligned with the second direction of transmission such that the insertion point of the auxiliary equipment will correspond to the target region of the subject shown in the ultrasonic image that has been captured by the ultrasonic medical imaging device being used.

2. The apparatus according to claim 1, wherein the soundwave deflection surface is configured to facilitate at least one of the following:—reflection, refraction, diffraction of sound waves.

3. The apparatus according to claim 1, wherein the soundwave deflecting surface is formed from a material having a sound transmission velocity at a ratio ranging from 1.0 to 11.0 relative to water.

4. The apparatus according to claim 1, wherein the soundwave manipulation module comprises a soundwave transmission portion arranged to facilitate transmission of the sound waves between at least the ultrasound medical imaging device and the soundwave deflection surface.

5. The apparatus according to claim 4, wherein the soundwave transmission portion is formed from a material having a sound transmission velocity at a ratio ranging from 0.8 to 5.0 relative to water.

6. The apparatus according to claim 4, wherein the soundwave transmission portion comprises a gel-based material.

7. The apparatus according to claim 6, wherein the gel-based material is disposed at a hollow portion of the sound wave manipulation module.

8. The apparatus according to claim 7, wherein the channel of the auxiliary equipment receiver is provided by a tubular channel extending through the hollow section.

9. The apparatus according to claim 8, wherein the gel-based material is accommodated within a gel pad that protrudes through a calibration window provided at a base plane of the apparatus.

10. The apparatus of claim 9, wherein the tubular channel extends to or through calibration window.

11. The apparatus according to claim 1, wherein the soundwave manipulation module is formed from a homogenous material having a sound transmission velocity at a ratio ranging from 1.0 to 3.0 relative to water.

12. The apparatus according to claim 11, wherein the sound wave manipulation module, the medical imaging device receiver and the auxiliary equipment receiver are integrally formed as a one-piece element.

13. The apparatus according to claim 1, comprising a handle for controlling movement of the apparatus on a body surface of the subject.

14. The apparatus according to claim 1, comprising a locking mechanism for maintaining the medical device at a desired position relative to a part of the apparatus.

15. A method for deploying the apparatus of claim 1 comprising the steps of:—
attaching an ultrasound medical imaging device to the medical imaging device receiver of the apparatus;
placing the apparatus on a body surface of a subject;
moving the apparatus on the body surface for obtaining an image of a target portion of the subject;
attaching an auxiliary equipment to the at least one auxiliary equipment receiver of the apparatus;
adjusting position of the auxiliary equipment along the pathway provided by the at least one auxiliary equipment receiver based on the image of the target portion, and inserting the auxiliary equipment towards the target portion.

16. A process of manufacturing an apparatus according to claim 9, comprising a process step of inserting the gel pad into the hollow section of the sound transmission module such that a portion of the gel pad protrudes through the calibration window.

17.-19. (canceled)
